(19) United States
(12) Patent Application Publication
(10) Pub. No.: US 2022/0313855 A1
(43) Pub. Date: Oct. 6, 2022

(54) ROBOTIC SYSTEMS FOR AUTONOMOUS TARGETED DISINFECTION OF SURFACES IN A DYNAMIC ENVIRONMENT AND METHODS THEREOF

(71) Applicant: EarthSense, Inc., Champaign, IL (US)

(72) Inventors: Joseph Byrnes, Champaign, IL (US); Michael Hansen, Champaign, IL (US); Chinmay Soman, Champaign, IL (US); Girish Chowdhary, Champaign, IL (US); Patric Liu, Champaign, IL (US)

(21) Appl. No.: 17/219,454

(22) Filed: Mar. 31, 2021

(51) Int. Cl. A61L 2/24; B25J 9/16; B25J 11/00

(52) U.S. Cl. CPC A61L 2/24; B25J 9/1697; B25J 9/1679; B25J 9/1664; B25J 11/0085; A61L 2202/14

(57) ABSTRACT

A method for managing autonomous targeted disinfection of surfaces in a dynamic environment includes determining one or more areas in an environment to target disinfection. A navigation path is generated based on at least layout data of the environment and the one or more determined areas in the environment to target disinfection. One or more drive system controls are generated and transmitted based on the generated navigation path to a robotic drive system. The one or more drive system controls to the robotic drive system are adjusted based on any obstacles during navigation that require an alteration of the generated navigation path. One or more disinfection arm system controls are initiated to guide positioning of a disinfection arm system to one of the areas to target disinfection when the one or more drive system controls have positioned the disinfecting arm system adjacent to the one of the areas.

### FIELD

This technology relates to robotic systems and methods for managing autonomous targeted disinfection of surfaces in a dynamic environment.

### BACKGROUND

The COVID19 pandemic is putting a serious strain on the healthcare system, and hospital-acquired infections can take healthcare personnel out of work (at best) and cause serious illness or death (at worst). Hospital admission also can put non-COVID19 patients at risk of acquiring the illness from other patients. Thorough disinfection of hospital rooms, clinics, and eldercare facilities can help reduce these infection rate, and high-intensity ultraviolet (UV) light is a promising approach for deep disinfection.

Although portable UV lamps and lamps mounted on mobile robots have been used successfully as illustrated in FIGS. 1A and 1B, these prior portable UV lamps and lamps mounted on mobile robots have some serious drawbacks. More specifically, occlusions to the UV light are caused by furniture, equipment, handles, crevices, and even buttons and dials, causing “shadow regions” that are not disinfected much, if at all. This means that UV disinfection has so far been limited to a precautionary backup to a first-round manual disinfection by wiping and scrubbing.

Another disadvantage is that UV disinfection of a room-scale environment from a central lamp is slow, requiring a room be vacated for up to an hour. This makes the technology inefficient for crisis situations. The slow operation rate is primarily due to radiant flux reduction at far distances according to the inverse square law. Unfortunately, these prior UV disinfection systems are unable to identify what areas need to be disinfected or to be able to position an UV emitter close enough to the surface to be disinfected so that the flux needed to inactivate pathogens can be delivered in under a minute, rather than tens of minutes.

### SUMMARY

A method for managing autonomous targeted disinfection of surfaces in a dynamic environment includes determining, by a computing device, one or more areas in an environment to target disinfection. A navigation path is generated, by the computing device, based on at least layout data of the environment and the one or more determined areas in the environment to target disinfection. One or more drive system controls are generated and transmitted, by the computing device, based on the generated navigation path to a robotic drive system. The one or more drive system controls to the robotic drive system are adjusted, by the computing device, based on any obstacles during navigation that require an alteration of the generated navigation path. One or more disinfection arm system controls are initiated, by the computing device, to guide positioning of a disinfection arm system to one of the areas to target disinfection when the one or more drive system controls have positioned the disinfecting arm system adjacent to the one of the areas.

A robotic system includes one or more sensor devices, a driving system, a disinfection arm system, and a management computing device. The management computing device is coupled to the one or more sensors, the driving system, and the disinfecting arm system and comprises a memory comprising programmed instructions stored thereon and one or more processors configured to be capable of executing the stored programmed instructions to determine one or more areas in an environment to target disinfection. A navigation path is generated based on at least layout data of the environment and the one or more determined areas in the environment to target disinfection. One or more drive system controls are generated and transmitted based on the generated navigation path to a robotic drive system. The one or more drive system controls to the robotic drive system are adjusted based on any obstacles during navigation that require an alteration of the generated navigation path. One or more disinfection arm system controls are initiated to guide positioning of a disinfection arm system to one of the areas to target disinfection when the one or more drive system controls have positioned the disinfecting arm system adjacent to the one of the areas.

A non-transitory computer readable medium having stored thereon instructions comprising executable code which when executed by one or more processors, causes the one or more processors to determine one or more areas in an environment to target disinfection. A navigation path is generated based on at least layout data of the environment and the one or more determined areas in the environment to target disinfection. One or more drive system controls are generated and transmitted based on the generated navigation path to a robotic drive system. The one or more drive system controls to the robotic drive system are adjusted based on any obstacles during navigation that require an alteration of the generated navigation path. One or more disinfection arm system controls are initiated to guide positioning of a disinfection arm system to one of the areas to target disinfection when the one or more drive system controls have positioned the disinfecting arm system adjacent to the one of the areas.

This technology provides a number of advantages including providing robotic systems and methods that manage autonomous targeted disinfection of identified surfaces in dynamic environments. Examples of this technology may utilize trained artificial intelligence software for navigation mapping and planning a disinfection path of both of the robotic system and of the arm-mounted disinfecting emitter for high intensity UV radiation, spraying, or a UV laser. Additionally, examples of this technology are able to selectively sanitize dynamic environments in the proximity of humans, eliminating a major limitation of prior full-room single-source UV radiation based robots that require the room to be unoccupied. Further, examples of this technology are able to radically increase the speed of disinfection in these dynamic environments, such as in hospitals, malls, offices, airports, and campuses by way of example only. With examples of this technology, the selective UV light exposure capability with the use of the arm-mounted disinfecting emitter alleviates prior concerns of over exposure with UV because the UV emitter can be directed to be close to the area requiring disinfection. Further examples of this technology unleash the tremendous promise UV has in improving sanitization while reducing costs through the minimization or elimination of cleaning chemicals.

### BRIEF DESCRIPTION OF THE DRAWINGS

FIGS. 1A-1B are perspective views of prior art UV disinfection robots;

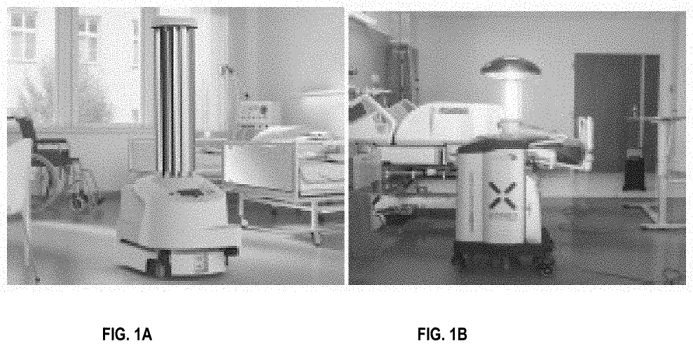

FIG. 2 is a perspective view of an example of an automated guided and targeted robotic disinfection system;

FIG. 3 is a block diagram of the example of the automated guided and targeted robotic disinfection system shown in FIG. 2;

FIG. 4 is a functional block diagram of an example of operation of the automated guided and targeted robotic disinfection system shown in FIG. 2;

FIG. 5 is a functional block diagram of an example of processing sensor data to generate navigation; and

FIG. 6 is a flowchart of an example of a method for managing autonomous targeted disinfection of one or more identified surfaces or other areas in a dynamic environment.

### DETAILED DESCRIPTION

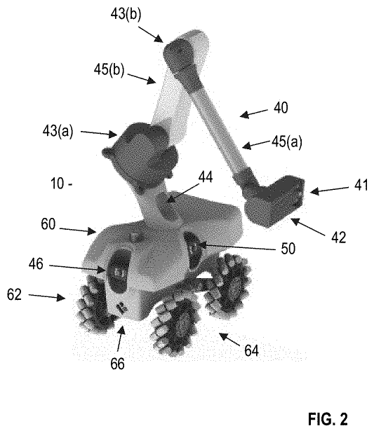

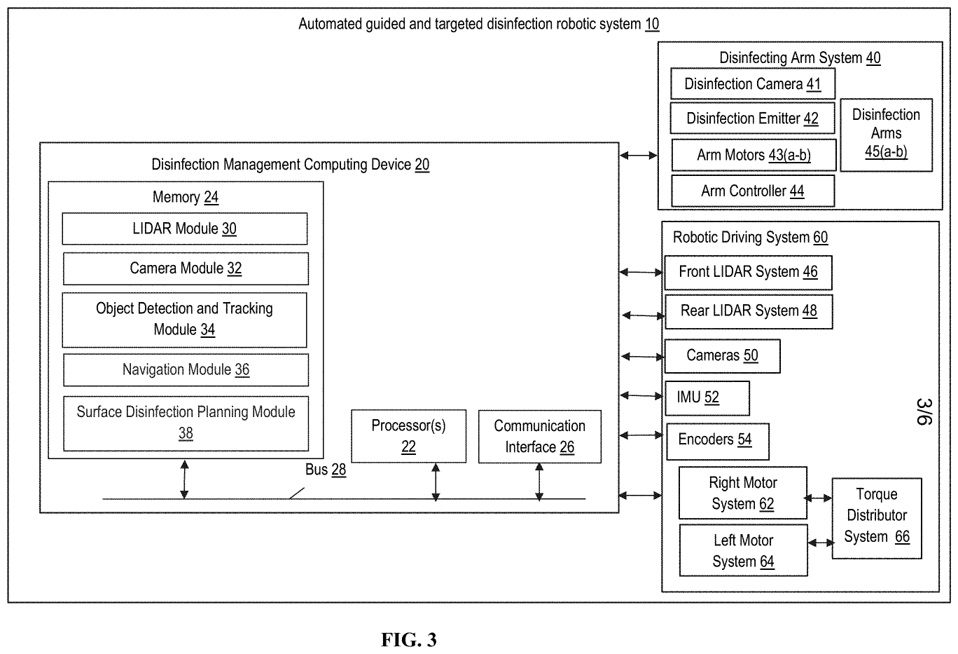

An exemplary automated guided and targeted robotic disinfection system 10 with a disinfection management computing device 20 is shown in FIGS. 2-3. In this example, the robotic disinfection system 10 includes a disinfection management computing device 20, a disinfecting arm system 40, and a robotic driving system 60, although the systems may comprise other types and/or numbers of other systems, devices, components, and/or other elements in other configurations. This technology provides a number of advantages including providing systems, methods, and non-transitory computer readable media that manage autonomous targeted disinfection of one or more identified surfaces or other areas in dynamic environments.

Referring to more specifically to FIGS. 2-3, in this example, the disinfecting arm system 40 comprises a disinfection camera 41, a disinfection emitter 42, arm motors 43(a) and 43(b), an arm controller 44, and disinfection arms 45(a) and 45(b), although the system 40 may comprise other types and/or numbers of other systems, devices, components and/or other elements in other configurations. The disinfection camera 41 is able to capture one or more images which may be used, by way of example, to identify particular surfaces for disinfection, identify any potential obstacles, such as individuals and/or objects by way of example, or other issues, and/or to guide positioning of the disinfection emitter 42, although other types and/or numbers of other imaging systems may be used and the disinfection camera 41 may be used to facilitate other operations. In this example, the disinfection camera 41 may comprise an RGB camera and an infra-red proximity sensor, although other types and/or numbers of imaging devices and/or other sensors may be used.

The disinfection emitter 42 is a high intensity UV light mounted on an end of adjustable arm 45(a) of the disinfecting arm system 40, although the disinfection emitter 42 may be at other locations on the disinfection arm system 40 and other types of disinfection emitters may be used, such as a UV laser and/or an embedded disinfectant sprayer by way of example only. In this particular example, the disinfection emitter 42 comprises 254 nm UVC light emitting diodes (LEDs), although emitters operating at other wavelengths for disinfection of a surface could be used. By way of another example, a 220 nm wavelength UVC emitter (which advantageously has only a very short range in biological material so that it cannot penetrate the dead-cell layer at, the surface of a person's skin or penetrate into a person's eye) could be used for the disinfection emitter 42. Additionally and unlike the prior art, the use of the guided and targeted UV emitter 42 prevents any individuals who are nearby from being exposed to significant amounts of UV radiation through the targeted application of UV.

The arm motors 43(a) and 43(b) are coupled to the arms 45(a) and 45(b), are under the control of the arm controller 44 based on one or more received commands from the disinfection management computing device 20, and may be engaged to rotate up to 360 degrees and/or move the arm(s) 45(a) and/or 45(b) in multiple directions to guide and position the disinfection emitter 42 to provide targeted disinfection of an identified surface, although other manners for providing guided and targeted positioning of one or more disinfection emitters may be used. In this particular example, an arm motor 43(a) is coupled between one end of arm 45(b) and the robotic driving system 20 and is configured to allow up to 360 degrees of rotation of arm 45(b) with respect to the robotic driving system 20 under the control of the arm controller 44 based on one or more commands from the disinfection management computing device 20, although other manners for managing the positioning of the arm 45(b) may be used. Additionally in this example, a motor 43(b) is coupled between ends of arms 45(a) and 45(b) and is used to manipulate the movement of arm 45(a) with respect to arm 45(b) under the control of arm controller 44 based on one or more commands from the disinfection management computing device 20, although other manners for managing the positioning of the arm 45(a) may be used. An additional motor may be coupled between the end of the arm 45(a) and the disinfection emitter 42, under the control of the arm controller 44 based on one or more commands from the disinfection management computing device 20, and used to adjust the position of the disinfection emitter 42, although other manners for managing the positioning of the disinfection emitter 42 may be used. With this disinfection arm system 40, the robotic system 10 is able to provide guided and targeted disinfection more safely and orders of magnitude faster than current broad area emission methods because of this ability to be precisely positioned close to identified areas or surfaces requiring disinfection.

The arm controller 44 is coupled to the disinfection emitter 42, the arm motors 43(a) and 43(b), and the disinfection management computing device 20 to manage and control movement and operation of the adjustable arms 45(a) and 45(b) and disinfection emitter 42 based on one or more commands from the disinfection management computing device 20 to provide guided and targeted disinfection, although the arm controller can execute other types and/or numbers of functions or other operations. For example, the arm controller 44 may also be configured to control motion of the adjustable arms 45(a) and 45(b) and the disinfection emitter 42 with the arm motors 43(a) and 43(b) based on one or more commands from the disinfection management computing device 20 to avoid contact with objects and/or individuals to prevent any damage or injuries. By way of example only, images from disinfection camera 41 may provide information on a current dynamic environment including any obstacles, such as any individuals and/or objects in the area, or other feedback data can be provided that the disinfection management computing device 20 can identify, analyze, and provide commands to the arm controller 44 to control movement of adjustable arms 45(a) and 45(b) and the disinfection emitter 42 to avoid any contact and/or maintain or achieve a desired positioning.

The adjustable arms 45(a) and 45(b) are pivotally coupled together at one end while another end of arm 45(b) is rotatably connected to the robotic driving system 20, although other types and/or numbers of arms or other disinfection emitter support or supports configured for connection and/or movement in other manners may be used. The particular dimensions of the adjustable arms 45(a) and 45(b) can vary as needed and in some examples one or both of the adjustable arms 45(a) and 45(b) could be designed to enable controlled adjustable extension or retraction in length. In this particular example, the arms 45(a) and 45(b) may include Quasi-Direct Drive (QDD) actuators based on low cost Brushless DC motors which are used of for motors 43(a) and 43(b) and are coupled to arm controller 44. The QDD actuators for motors 43(a) and 43(b) are well suited for working in close proximity to obstacles, such as individuals and/or objects by way of example, due to their back drivability, selectable impedance, and robust force control to enable control over positioning of arms 45(a) and 45(b) to avoid harm to these obstacles through accidental contact. The QDD actuators for motors 43(a) and 43(b) also enable providing current and position feedback for the disinfection management computing device 20 to be able to determine when arms 45(a) and/or 45(b) may have collided with a surface without the need for expensive torque sensors placed at the output of the actuators. With a programmed dynamic model of the robot arms 45(a) and 45(b) and compensations for friction and gravity, the disinfection management computing device 20 may also precisely determine how much torque each of the QDD actuators for motors 43(a) and 43(b) need to exert to maintain their current position assuming no contact with obstacles, such as individuals and/or objects by way of example. Upon sensing that more torque is required to keep the arms 45(a) and 45(b) in position, the disinfection management computing device 20 may determine there has been contact with an obstacle and be able to issue commands to the arm controller 44 to react safely by not only compliantly pushing out of the way, but also by halting and changing the motion plan.

The robotic driving system 60 includes all of the parts of a motor vehicle system including, by way of example, a body, engine, fuel system, steering system, brake system, powertrain, and wheels and is used to drive the robotic system 10 in the dynamic environment, although other types of systems to enable movement of the robotic system 10 may be used. In this particular example, the robotic driving system 60 has right and left motor systems 62 and 64 which are coupled to a torque distributor system 66 that is driven by powertrain powered by a motor coupled to a fuel source, such as a battery by way of example, and whose operation is managed by a motor controller, such as disinfection management computing device 20 by way of example only, although other types and/or numbers of systems, devices, components and/or other elements to enable guided motorized movement of the robotic system 10 in the dynamic environment may be used. Additionally, in this example the robotic drive system 60 may have four independently controlled wheels mounted on a suspension to ensure continuous and evenly distributed contact with a surface of the dynamic environment. By way of example only, an exemplary robotic driving system or vehicle which could be used in examples here is illustrated and described by way of example in WO 2019/040866, which is herein incorporated by reference in its entirety.

To enhance balance, the robotic driving system 60 may arrange components of the motor system which are heavier towards the bottom of a housing for the robotic driving system 60, such as the battery or other power or fuel source by way of example. By concentrating the weight near the bottom, any center of gravity movement is limited as the arms 45(a) and 45(b) are rotated, extended, or otherwise positioned. Additionally, the ground-clearance of the robotic driving system 60 may be reduced, and the suspension stiffened, to lower the center of gravity of the robotic system 10 and increase stability. Further and by way of example only, the robotic system 10 with this robotic drive system 60 may have a length of about 21.5 inches and a width of about 12 inches to minimize the overall footprint of the robotic system 10 and enhance maneuverability, although the robotic system 10 could have other dimensions depending on the particular dynamic environment

The robotic driving system 60 may use an omnidirectional drive system to minimize disturbance to obstacles, such as individuals moving past the robotic system 10 in a dynamic environment, and maximize the reachable workspace of the robotic system 10 without the need for complex and time consuming maneuvers. Additionally, the robotic driving system 60 may, in this particular example, comprise a Mecanum drive system with Mecanum wheels which is able to move in any direction without the need to change orientation before or while moving, although other types of drive systems may be used. Accordingly, this Mecanum drive system shortens the time required for the robotic drive system 60 to react to the dynamic obstacles found the environment which is advantageous.

Additionally, in this particular example the front and rear light detection and ranging (LIDAR) systems 46-48, the cameras 50, the inertial measurement unit (IMU) 52, and the encoders 54 may all be housed within the robotic driving system 60, although one or more of these systems, devices, components or other elements could be at other locations in other examples. The robotic driving system 60 may also comprise or otherwise house or support other types and/or numbers of other systems, devices, components, and/or other elements in other configurations. The front and rear light detection and ranging (LIDAR) systems 46-48, the cameras 50, the inertial measurement unit (IMU) 52, and the encoders 54 are each coupled to the disinfection management computing device 20, although each may have other types and/or numbers of connections to other systems, devices, components and/or other elements to enable the automated guided and targeted disinfection as illustrated and described by way of the examples herein.

In this example, the front LIDAR and rear system 46 and 48 are each light detection and ranging systems which are located on opposing ends of a housing for the robotic driving systems and the three cameras 50 are imaging systems which are positioned about the robotic diving system 60 to capture different types of imaging data, although other types and/or numbers of imaging systems may be used, such as a fish eye camera and/or a thermal imaging system by way of example only. In other examples, the one or more cameras 50 and/or one or more of the other sensors and systems may comprise a depth sensing system that is capable of capturing imaging and other data, such as depth data, that may be analyzed to measure and obtain depth information. By way of example only, the camera(s) 50 in a depth sensing system may be Intel RealSense sensors or stereo sensors. The depth sensing data may be used by the disinfection management computing device 20 to manage generation of instructions for navigation of the robotic system 10 and/or for managing automated guided and targeted disinfection with the disinfecting arm system 40, although the depth sensing data may be used for other types and/or numbers of operations used to manage the robotic system 10.

In this example, the inertial measurement unit (IMU) 52 is in the robotic driving system 60, is coupled to the disinfection management computing device 20, and may measure and report data, such as a specific force, angular rate, and orientation of the robotic system 10 in this example using a combination of accelerometers, gyroscopes, and/or magnetometers, although other types and/or numbers of measurement devices may be used by the robotic system 10. Additionally, the encoders 54 are in the robotic driving system 60, are coupled to the disinfection management computing device 20, and may comprise one or more sensors that provide feedback on operational characteristics of the robotic system 10, such as a motion or position of one or more aspects of the robotic system 10 by way of example, although other types and/or numbers of measurement systems may be used.

The disinfection management computing device 20 is coupled to the disinfection arm system 40 and the robotic driving system 10 and may execute any number of functions and/or other operations to manage autonomous targeted disinfection of one or more identified surfaces or other areas in dynamic environments as illustrated and described by way of the examples herein. In this particular example, the disinfection management computing device 20 includes one or more processor(s) 22, a memory 24, and/or a communication interface 26, which are coupled together by a bus or other communication link 28, although the disinfection management computing device 20 can include other types and/or numbers of elements in other configurations.

The processor(s) 22 of the disinfection management computing device 20 may execute programmed instructions stored in the memory of the disinfection management computing device 20 for any number of functions and other operations as illustrated and described by way of the examples herein. The processor(s) 22 of the disinfection management computing device 20 may include one or more CPUs or general purpose processors with one or more processing cores, for example, although other types of processor(s) can also be used.

The memory 24 of the disinfection management computing device 20 stores these programmed instructions for one or more aspects of the present technology as described and illustrated herein, although some or all of the programmed instructions could be stored elsewhere. A variety of different types of memory storage devices, such as random access memory (RAM), read only memory (ROM), hard disk, solid state drives, flash memory, or other computer readable medium which is read from and written to by a magnetic, optical, or other reading and writing system that is coupled to the processor(s), can be used for the memory 24.

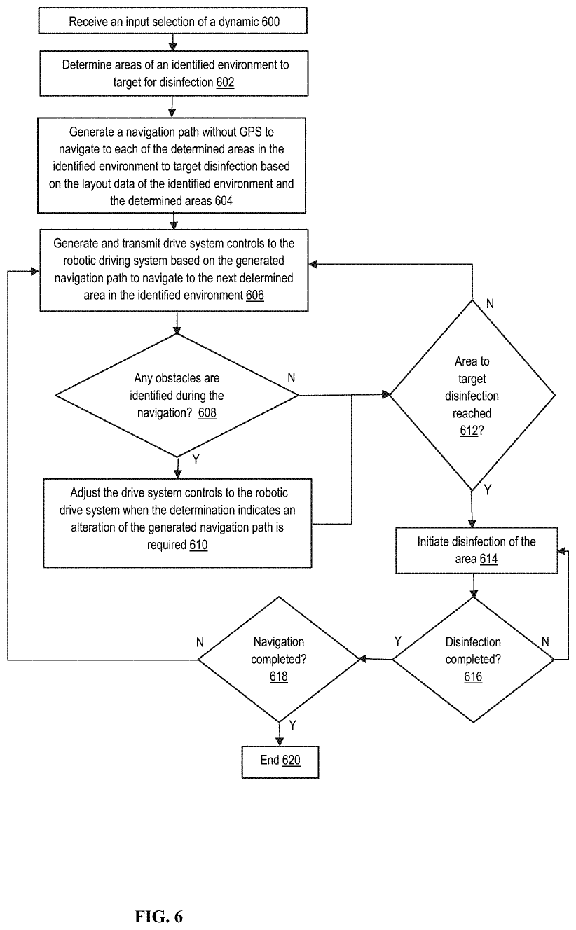

Accordingly, the memory 24 of the disinfection management computing device 20 can store one or more applications that can include computer executable instructions that, when executed by the disinfection management computing device 20, cause the disinfection management computing device 20 to perform actions, such as to manage autonomous targeted disinfection of one or more identified surfaces or other areas in a dynamic environment, and other actions as described and illustrated in the examples below with reference to FIGS. 2-6. The application(s) can be implemented as modules, programmed instructions or components of other applications. Further, the application(s) can be implemented as operating system extensions, module, plugins, or the like.

Even further, the application(s) may be operative in a cloud-based computing environment coupled to the robotic system 10. The application(s) can be executed within or as virtual machine(s) or virtual server(s) that may be managed in a cloud-based computing environment. Also, the application(s), and even the disinfection management computing device 20 itself, may be located in virtual server(s) running in a cloud-based computing environment rather than being tied to one or more specific physical computing devices in the robotic system 10. Also, the application(s) may be running in one or more virtual machines (VMs) executing on the disinfection management computing device 20. Additionally, in one or more embodiments of this technology, virtual machine(s) running on the disinfection management computing device 20 may be managed or supervised by a hypervisor.

In this particular example, the memory 24 of the disinfection management computing device 20 may include a LIDAR module 30, a camera module 32, an object detection and tracking module 34, a navigation module 36, and a surface disinfection planning module 38 which may be executed as illustrated and described by way of the examples herein, although the memory 24 can for example include other types and/or numbers of modules, platforms, algorithms, programmed instructions, applications, or databases for implementing examples of this technology.

The LIDAR module 30 and camera module 32 may comprise executable instructions that are configured to process imaging data captured by the front and rear LIDAR systems 46 and 48 and the cameras 50 as illustrated and described in greater detail by way of the examples herein, although each of these modules may have executable instructions that are configured to execute other types and/or functions or other operations to facilitate examples of this technology.

Additionally in this example, the object detection and tracking module 34 may comprise executable instructions that are configured to identify and track any obstacles in the processed imaging data captured by the front and rear LIDAR systems 46 and 48 and/or the cameras 50 as illustrated and described in greater detail by way of the examples herein, although this module may have executable instructions that are configured to execute other types and/or functions or other operations to facilitate examples of this technology. In these dynamic environments, the overall layout may remain the same, but location of obstacles, such as individuals and/or objects, may dynamically change.

The navigation module 36 may comprise executable instructions that are configured to enable autonomous navigation of the robotic system 10 without use of a global position system (GPS) and which adjust to the dynamic environment as one or more obstacles, such as individuals and/or objects, are identified as illustrated and described in greater detail by way of the examples herein, although this module may have executable instructions that are configured to execute other types and/or functions or other operations to facilitate examples of this technology. The navigation module 36 may also comprise executable instructions that are configured to process prior stored target data on one or more areas in the identified environment or other environments determined to be related to the identified environment based on one or more factors, such as types or categories of environments by way of example only, to identify one or more areas which require disinfection and generate intelligent arm motion planning for the disinfection arm system 40 for precise UV light disinfection with the arm-mounted ultraviolet (UV) emitter 42 of those areas. Further, the surface disinfection planning module 38 also may comprise executable instructions that are configured to generate other types of control instructions for the disinfection arm system 40, such as based on current and position feedback data provided from when arms 45(a) and/or 45(b) may have collided with a surface or other object

In this particular example, the navigation module 36 comprises executable instructions for one or more Simultaneous Localization and Navigation mapping (SLAM) algorithms for reliable and non-intrusive autonomous operation in dynamic environments which may comprises crowded places because of obstacles, such as individual(s) and/or obstacle(s) by way of example only. This example of the navigation module 36 when executed by the disinfection management computing device 20 leverages environmental imaging data obtained from the front and rear LIDAR systems 46 and 48 and cameras 50 on the robotic driving system 60 augmented with Mecanum wheels that improve maneuverability in tight spots to generate and provide driving control instructions to the robotic driving system 60 and may provide arm operation instructions to arm controller 44, such as arm operation instructions to keep arms 45(a) and 45(b) at least partially retracted until the surface to disinfect is reached by way of example only. The navigation module 36 may also utilize inputs from other sources, such as from the IMU 52 and/or encoders 54 by way of example, when generating driving control instructions for the robotic driving system 60 and/or arm control operation instructions for the disinfection arm system 40. Accordingly, this example of the navigation module 36 is capable of processing detecting, and providing driving control instructions to manage and also avoid dynamic obstacles, such as moving individuals, equipment, furniture, and/or other objects that may have changed locations, with the disinfecting arm system 40 and robotic driving system 60.

In this particular example, the navigation module 36 does not use and the robotic system 10 does not have a global positioning system (GPS) because GPS does not work well in areas where direct visibility of the sky is compromised, such as indoor environments or when a GPS signal is jammed. In other examples, GPS or other systems which simulate or otherwise facilitate use of GPS could be used by the navigation module 36 to manage or assist navigation of the robotic system 10. Instead the robotic system 10 uses a combination of exteroceptive sensors, such as LIDAR LIDAR systems 46-48, cameras 50, and proprioceptive sensors such as inertial measurement unit (IMU) 52 and wheel encoders 54 for navigation. The robotic system 10 may employ simultaneous localization and mapping (SLAM) techniques for autonomous navigation in the indoor environment in this example. The layout data or map(s) of the environment may be augmented using prior geometric information of the environment. In other examples, the layout data or map(s) of the environment may be further corrected by similarity detection of the current layout data to layout data of other correlated layout data of one or more other different places to find similar places or objects and remove ambiguities. The similarity detection may be accomplished using convolutional neural networks by way of example only.

The surface disinfection planning module 38 may comprise executable instructions that are configured to determine one or more areas of an identified environment to target disinfection and generate intelligent arm motion planning controls for the disinfection arm system 40 for precise UV light disinfection with the arm-mounted ultraviolet (UV) emitter 42 as illustrated and described in greater detail by way of the examples herein, although this module may have executable instructions that are configured to execute other types and/or functions or other operations to facilitate examples of this technology. By way of example, the surface disinfection planning module 38 may comprise executable instructions that are configured to process tracked traffic and/or physical contact data in the identified environment to identify one or more areas in the identified environment to disinfect, although other manners for identifying the areas may be used. By way of another example, the surface disinfection planning module 38 may comprise executable instructions that are configured to process to imaging data from disinfection camera 41 to dynamically identify one or more areas which require disinfection and generate intelligent arm motion planning for the disinfection arm system 40 for precise UV light disinfection with the arm-mounted ultraviolet (UV) emitter 42 of those areas. In other examples, other types of disinfection techniques may be used, such as spraying of disinfectants or even wiping the contaminated area by way of example.

The communication interface 26 of the disinfection management computing device 20 operatively couples and communicates between the disinfection management computing device 20 and the disinfection arm system 40 and robotic driving system 60, which are all coupled together, although other types and/or numbers of connections and/or communication networks can be used.

While the disinfection management computing device 20 is illustrated in this example as including a single device, the disinfection management computing device 20 in other examples can include a plurality of devices each having one or more processors (each processor with one or more processing cores) that implement one or more steps of this technology. In these examples, one or more of the devices can have a dedicated communication interface or memory. Alternatively, one or more of the devices can utilize the memory, communication interface, or other hardware or software components of one or more other devices included in the disinfection management computing device 20.

Additionally, one or more of the devices that together comprise the disinfection management computing device 20 in other examples can be standalone devices or integrated with one or more other devices or apparatuses, such as in one of the server devices or in one or more computing devices for example. Moreover, one or more of the devices of the disinfection management computing device 20 in these examples can be in a same or a different communication network including one or more public, private, or cloud networks, for example.

Although an exemplary disinfection management computing device 20 is described and illustrated herein, other types and/or numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the components depicted in this robotic disinfection system 10, such as the disinfection management computing device 20, for example, may be configured to operate as virtual instances on the same physical machine. In other words, by way of example one or more of the disinfection management computing device 20 may operate on the same physical device rather than as separate devices communicating through communication network(s). Additionally, there may be more or fewer disinfection management computing device 20 than illustrated in FIG. 3.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) that extend across any suitable network using any suitable interface mechanisms and traffic technologies, including by way of example only teletraffic in any suitable form (e.g., voice and modem), wireless traffic networks, cellular traffic networks, Packet Data Networks (PDNs), the Internet, intranets, and combinations thereof.

The examples may also be embodied as one or more non-transitory computer readable media having instructions stored thereon for one or more aspects of the present technology as described and illustrated by way of the examples herein. The instructions in some examples include executable code that, when executed by one or more processors, cause the processors to carry out steps necessary to implement the methods of the examples of this technology that are described and illustrated herein.

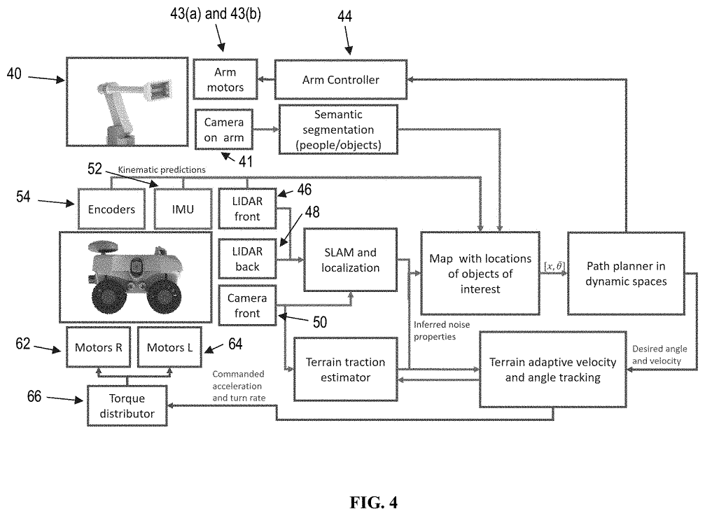

An exemplary method for managing autonomous targeted disinfection of one or more identified surfaces or other areas in a dynamic environment with the robotic disinfection system 10 will now be described with reference to FIGS. 2-6. Referring more specifically to FIGS. 4-6, in this example in step 600, the disinfection management computing device 20 may receive a selection or other input of a dynamic environment to disinfect. In this example, the disinfection management computing device 20 may retrieve location or other layout data for the environment identified for disinfection, although other manners for obtaining layout data of the environment identified for disinfection may be used. In another example, in a setup stage the robotic disinfection system 10 may be initially guided by an operator through commands input to the disinfection management computing device 20 to capture and/or update layout data of a representation of the identified environment to be used for later automated navigation and guided and targeted disinfection, although other manners for obtaining the layout data may be used.

In step 602, the disinfection management computing device 20 may execute the surface disinfection planning module 38 to determine one or more areas of an identified environment to target disinfection, although other manners for determining the areas to disinfect may be used. In this example, the disinfection management computing device 20 may obtain and process current and/or historical monitored traffic data and/or monitored physical contact data in the environment to identify the one or more determined areas in the identified environment to disinfect, although other types of data may be obtained and processed to determine areas to disinfect may be obtained. In another example, the disinfection management computing device 20 may capture imaging data with the disinfection camera 41 in the disinfection arm system 40 during an initial scouting of the environment to obtain layout data of the environment and/or during the navigation which can be used to dynamically determine one or more areas in the identified environment to target disinfection and/or to update one or more previously determined areas to target disinfection, such as to add, remove or resize one or more of the areas to target disinfection by way of example only.

In another example, the disinfection management computing device 20 may execute an artificial intelligence target area identification algorithm based on the obtained imaging data, such as imaging data from the disinfection camera 41 by way of example only, to identify or update the one or more determined areas. This artificial intelligence target area identification algorithm may be trained based on prior stored imaging data and associated determined areas in the identified environment or one or more other environments determined to be comparable to the identified environment for areas for disinfection based on one or more factors, such as similarities in type and size of the environments by way of example only. By way of example, comparable environments correlated to the current environment may have similarities in general areas with one or more related areas that require disinfection, such as touch surface areas at initial check in locations, bathroom door handles, hand rails, elevator button panels, handles of medical equipment, etc. In other examples, the determined areas may identify locations for the robotic system 10 to travel to and then to determine based on analysis of imaging particular determined areas or surfaces to disinfect.

In step 604 the disinfection management computing device 20 may execute one or more Simultaneous Localization and Navigation mapping (SLAM) algorithms in the navigation module 36 that comprise executable instructions to generate a navigation path to navigate to each of the determined one or more areas in the identified environment to target disinfection based on the layout data of the identified environment and the one or more determined areas without the use of GPS, although other manners for managing navigation may be used. In this example, when determining the navigation path, the one or more Simultaneous Localization and Navigation mapping (SLAM) algorithms also may determine: how a kinematic model the robot system 20, including the position and extension or retraction of the disinfecting arm system 40, may be configured to avoid collision with the geometry of and any obstacles in the identified environment while navigating; how long the robotic system 10 is in different locations in the environment to ensure areas, such as particular surfaces by way of example, are exposed to sufficient UV light; and how the robotic system 10 is configured to scan for unseen areas that need disinfection, although other types and/or numbers of other best view and coverage-based path planning and disinfection management techniques may be used.

In step 606 the disinfection management computing device 20 may generate and transmit one or more drive system controls to the robotic driving system 60 based on the generated navigation path to sequentially navigate to the one or more determined areas in the identified environment to target disinfection. The disinfection management computing device 20 also may generate and transmit one or more navigation arm system controls to the disinfecting arm control system 40 to motors 43(a-b) to adjust positioning of one or more arms 45(a-b) of the disinfection arm control system 40 to facilitate the navigation, although other manners for adjusting the disinfecting arm system 40, such as the positioning and extension or retraction of the arms 45(a-b) may be used.

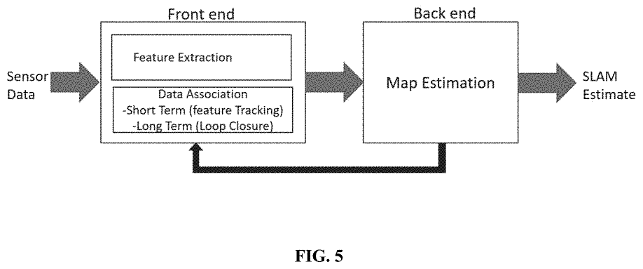

In this example, as the robotic system 10 navigates the path, the disinfection management computing device 20 may extract visual feature key-points from imaging or other data provided by, for example, front and rear LIDAR 46-48 and front camera 50 for the purpose of estimating motion and position of the robotic system 10 in the environment, although other captured data, such as from IMU 52 and/or encoders 54 by way of example, and other manners for managing navigation may also be used. Additionally, in this example the disinfection management computing device 20 during navigation may determine and apply and necessary corrections for any drift or other navigation errors based on monitoring the progress of the robotic system 10 along the navigation path in the environment. Further, an example of processing sensor data from, for example, front and rear LIDAR 46-48, front camera 50, IMU 52 and/or encoders 54, for the SLAM algorithm that may include feature extraction from the data and short term and long term data association to generate or modify drive system controls for navigation is illustrated in FIG. 5.

In step 608, the disinfection management computing device 20 may determine when any obstacles are identified by front and rear LIDAR 46-48 and a camera 50 during the navigation, although other manners for identifying obstacles may be used. In a dynamic environment, objects, such as people and/or equipment, may be constantly changing. In this example, the disinfection management computing device 20 executing the one or more Simultaneous Localization and Navigation mapping (SLAM) algorithms in the navigation module 36 for enabling autonomous navigation of the robotic system 10 in the environment may also be solving the simultaneous problem of localizing the robotic system 10 with respect to the navigation path in the environment, as well as keeping the navigation path of the environment and/or the layout data of the environment updated based on any identified obstacles. Additionally, in this example the one or more Simultaneous Localization and Navigation mapping (SLAM) algorithms also may comprises a number of sensor-fusion techniques that fuse data from multiple sensors, such as front and rear LIDAR 46-48, front camera 50, IMU 52, and/or encoders 54 by way of example only, to estimate the pose (position and attitude) of the robotic system 10 with respect to the navigation path to manage navigation. If in step 608 the disinfection management computing device 20 determined an obstacle is identified, then the Yes branch is taken to step 610.

In step 610, the disinfection management computing device 20 may adjust the one or more drive system controls to the robotic driving system 60 based on one or more obstacles identified by one or more imaging devices during the navigation that trigger an alteration of the generated navigation path. For example, the disinfection management computing device 20 may identify any obstacles based on received data from one or more sources, such as front and rear LIDAR 46-48, front camera 50, IMU 52, and/or encoders 54 by way of example only, during navigation of the generated navigation path during the navigation. Next, the disinfection management computing device 20 may determine when any of the identified obstacles during the navigation require an alteration of the generated navigation path and then accordingly adjust the one or more drive system controls to the robotic drive system.

By way of example, a hospital environment is inherently a dynamic setting with patients, equipment, furniture, and other objects moving or being moved, and with medical staff going from one room to another. These dynamic changes in the environment also make it difficult for the robotic system 10 to rely on a pre-planned path. Accordingly, the disinfection management computing device 20 may readjust continuously the navigation path and resulting generated drive control signals based on a form of a dynamically updated occupancy grid in this example. Another aspect of examples of this technology is that the disinfection management computing device 20 in the robotic disinfection system 10 performs these adjustment operations in real-time while navigating.

When determining how to adjust the drive system controls, the disinfection management computing device 20 may also track any objects in view, determine whether any of the objects are moving, and then determine what the likely trajectory of each of those objects is which is then factored in to adjust the driving control signals to manage dynamic navigation. By way of example only, the disinfection management computing device 20 may add a predictive module which when executed on consecutive image frame data predicts future movement of a tracked object and may use a Bayesian predict-correct algorithm in a Kalman filtering framework to improve on-going tracking of the object.

The disinfection management computing device 20 may also generate and transmit one or more navigation arm system controls to the disinfecting arm control system to adjust one or more arms 45(a-b) of the disinfection arm control system 40 to a retracted or other position to facilitate the navigation and/or avoid an identified object. When in motion, the disinfection management computing device 20 may for example retract one or more arms 45(a-b) of the disinfection arm control system 40 to a stable view from where the arm mounted disinfection camera 41 can look forward. The object detection and tracking module 34 executed by the disinfection management computing device 20 may ingest images from the arm mounted camera to detect objects in the view. Traditional neural networks, such as YOLO or faster-RCNN trained on benchmark datasets, can be trained on prior object detection data and then may be used by the disinfection management computing device 20 to enhance the accuracy of the detection of objects in this manner.

Next, either following step 610 or if back in step 608 the disinfection management computing device 20 determined an obstacle is not identified so that the No branch is taken, then this example proceeds to step 612. In step 612 the disinfection management computing device 20 may determine if the next area to target disinfection in the environment has been reached. If in step 612 the disinfection management computing device 20 determines the next area to target disinfection in the environment has not been reached, then the No branch is taken back to step 606 as described earlier. If in step 612 the disinfection management computing device 20 determines the next area to target disinfection in the environment has been reached, then the Yes branch is taken to step 614.

In step 614, the disinfection management computing device 20 may initiate one or more disinfection arm system controls to guide extension and positioning of arms 45(a) and 45(b) with arm motors 43(a-b) of disinfection arm system 40 to the one of the areas to target disinfection when the drive system controls have positioned the disinfecting arm system 40 adjacent to the one of the areas. The particular area(s) to disinfect may be obtained or identified in a variety of different manners, such as from an initial scouting of the environment which identified the one or more particular areas or surfaces, from historical or other input of data identifying the particular areas or surfaces, and/or dynamically from captured imaging or other scanned and analyzed data by the robotic system 60 identifying the particular areas or surfaces to disinfect by way of example. The disinfection management computing device 20 may determine from captured imaging, such as with a depth sensing system from camera(s) 50 by way of example only, particular location, depth and other positioning data of each of the particular areas or surfaces to disinfect. This particular location, depth and other positioning data of each of the particular areas or surfaces to disinfect can be used by the disinfection management computing device 20 to generate arm control instructions for disinfecting arm system 40 to position the emitter 42 using arms 45(a-b) in this example for targeted disinfection without exposing the entire area to UV. In this example, the disinfection management computing device 20 may target disinfection of the area using the disinfection emitter 42 with targeted ultraviolet light, although other types and/or numbers of other targeted disinfection system can be used, such as engaging a chemical spray disinfection or other disinfection treatment with the disinfecting arm system 40.

In step 616, the disinfection management computing device 20 may determine if the disinfection of the area has been completed. By way of example, the disinfection management computing device 20 may monitor the targeted disinfection of the one of the areas with, by way of example only, one or more of the front LIDAR 46, rear LIDAR 48, front camera 50, IMU 52, and/or encoders 54 by way of example only, and analyze the captured data to determine when the targeted disinfection to the one areas is completed, although other manners for determining when disinfection is completed may be used, such as based on a monitored length of time for the disinfection cycle by way of example.

If in step 616 the disinfection management computing device 20 determines the targeted disinfection to the one areas is not completed, then the No branch is taken back to step 614 to continue the disinfection process with the targeted disinfection using the disinfection emitter 42 with ultraviolet light in this example. If in step 616 the disinfection management computing device 20 determines the targeted disinfection to the one areas is completed, then the Yes branch is taken back to step 618.

In step 618, the disinfection management computing device 20 determines when the navigation through and disinfection of all of the determined areas, such as previously identified areas and/or areas identified during navigation through the environment, is completed. If in step 618 the disinfection management computing device 20 determines the targeted disinfection of all the areas is not completed, then the No branch is taken back to step 606 to generate and transmit system control to the robotic drive system 60 to navigate to the next determined area. If in step 618 the disinfection management computing device 20 determines the targeted disinfection of all the areas is completed, then the Yes branch is taken to step 620 where this example of the method may end.

Accordingly, as illustrated and described by way of the examples herein this technology enables providing robotic systems and methods that manage autonomous targeted disinfection of identified surfaces in dynamic environments. Examples of this technology may utilize trained artificial intelligence software for navigation mapping and planning a disinfection path of both of the robotic system and of the arm-mounted disinfecting emitter for high intensity UV radiation, spraying, or a UV laser. Additionally, examples of this technology are able to selectively sanitize dynamic environments in the proximity of humans, eliminating a major limitation of prior full-room single-source UV radiation based robots that require the room to be unoccupied. Further, examples of this technology are able to radically increase the speed of disinfection in these dynamic environments, such as in hospitals, malls, offices, airports, and campuses by way of example only. With examples of this technology, the selective UV light exposure capability with the use of the arm-mounted disinfecting emitter alleviate prior concerns of over exposure with UV by placing the UV emitter close to the area. Even further examples of this technology unleash the tremendous promise UV has in improving sanitization while reducing costs through the minimization or elimination of cleaning chemicals.

Having thus described the basic concept of the invention, it will be rather apparent to those skilled in the art that the foregoing detailed disclosure is intended to be presented by way of example only and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method comprising:
determining, by a computing device, one or more areas in an environment to target disinfection;
generating, by the computing device, a navigation path based on at least layout data of the environment and the one or more determined areas in the environment to target disinfection;
generating and transmitting, by the computing device, one or more drive system controls based on the generated navigation path to a robotic drive system;
adjusting, by the computing device, the one or more drive system controls to the robotic drive system based on any obstacles during navigation that require an alteration of the generated navigation path; and
initiating, by the computing device, one or more disinfection arm system controls to guide positioning of a disinfection arm system to one of the areas to target disinfection when the one or more drive system controls have positioned the disinfecting arm system adjacent to the one of the areas.

2. The method as set forth in claim 1 wherein the determining one or more areas in the environment to target disinfection further comprises:
obtaining, by the computing device, at least one of monitored traffic data or monitored physical contact data in the environment; and
processing, by the computing device, the at least one of the monitored traffic data or the monitored physical contact data in the environment to identify the one or more determined areas in the identified environment to disinfect.

3. The method as set forth in claim 2 wherein the determining one or more areas in the environment to target disinfection further comprises:
obtaining, by the computing device, imaging data from at least one of the imaging devices during the navigation; and
processing, by the computing device, the imaging data from to at least one of identify or update the one or more determined areas.

4. The method as set forth in claim 3 wherein the processing the imaging data to at least one of identify or update the one or more determined areas further comprises:
executing, by the computing device, an artificial intelligence target area identification algorithm based on the obtained imaging data to at least one of identify or update the one or more determined areas;
wherein the artificial intelligence target area identification algorithm is trained based on prior stored imaging data and associated determined areas in at least one of the environment or one or more other environments determined to be comparable to the environment based on one or more factors.

5. The method as set forth in claim 3 wherein the at least one of the imaging devices comprises at least one camera mounted on the disinfecting arm system and capable of measuring at least depth information.

6. The method as set forth in claim 1 wherein the generating and transmitting the one or more drive system controls based on the generated navigation path to a robotic drive system further comprises:
generating and transmitting one or more navigation arm system controls to the disinfecting arm control system to adjust positioning of one or more arms of the disinfection arm control system.

7. The method as set forth in claim 1 wherein the adjusting the one or more drive system controls to the robotic drive system further comprises:
receiving navigation imaging data from one or more imaging devices during navigation of the generated navigation path;
identifying any obstacles in the navigation imaging data during the navigation;
determining when any of the identified obstacles during the navigation require an alteration of the generated navigation path;
creating updated layout data of the environment with a stored relative position of the identified objects; and
adjusting the one or more drive system controls to the robotic drive system based on the updated layout data of the environment with the stored relative position of the identified objects to create one or more alterations to the navigation path when the determination indicates an alteration of the generated navigation path is required.

8. The method as set forth in claim 7 wherein the one or more imaging devices comprise an imaging and depth sensing system with at least one LIDAR and one or more cameras that capture imaging and depth sensing data; and
wherein at least one of the adjusting the one or more drive system controls to the robotic drive system or generating and transmitting one or more navigation arm system controls to the disinfecting arm control system to adjust positioning of one or more arms of the disinfection arm control system is based on the imaging and depth sensing data.

9. The method as set forth in claim 1 further comprising:
monitoring, by the computing device, the targeted disinfection to the one of the determined areas; and
determining, by the computing device, when the targeted disinfection to the one determined areas is completed;
wherein the generating and transmitting the one or more drive system controls further comprises generating and transmitting one or more additional drive controls to navigate the generated navigation path to a next one of the determined areas in the environment when the determination indicates the targeted disinfection to the one areas is completed.

10. A robotic system, the system comprising:
one or more sensor devices;
a driving system;
a disinfection arm system;
a management computing device coupled to the one or more sensors, the driving system, and the disinfecting arm system and comprising a memory comprising programmed instructions stored thereon and one or more processors configured to be capable of executing the stored programmed instructions to:
determine one or more areas in an environment to target disinfection;
generate a navigation path based on at least layout data of the environment and the one or more determined areas in the environment to target disinfection;
generate and transmit one or more drive system controls based on the generated navigation path to a robotic drive system;
adjust the one or more drive system controls to the robotic drive system based on any obstacles during navigation that require an alteration of the generated navigation path; and
initiate one or more disinfection arm system controls to guide positioning of a disinfection arm system to one of the areas to target disinfection when the one or more drive system controls have positioned the disinfecting arm system adjacent to the one of the areas.

11. The system as set forth in claim 10 wherein the one or more processors are further configured to be capable of executing the stored programmed instructions to:
obtain at least one of monitored traffic data or monitored physical contact data in the environment; and
process the at least one of the monitored traffic data or the monitored physical contact data in the environment to identify the one or more determined areas in the identified environment to disinfect.

12. The system as set forth in claim 11 wherein the one or more processors are further configured to be capable of executing the stored programmed instructions to:
obtain imaging data from at least one of the imaging devices during the navigation; and
process the imaging data from to at least one of identify or update the one or more determined areas.

13. The system as set forth in claim 12 wherein for the process the imaging data to at least one of identify or update the one or more determined areas, the one or more processors are further configured to be capable of executing the stored programmed instructions to:
execute an artificial intelligence target area identification algorithm based on the obtained imaging data to at least one of identify or update the one or more determined areas;
wherein the artificial intelligence target area identification algorithm is trained based on prior stored imaging data and associated determined areas in at least one of the environment or one or more other environments determined to be comparable to the environment based on one or more factors.

14. The method as set forth in claim 12 wherein the at least one of the imaging devices comprises at least one camera mounted on the disinfecting arm system and capable of measuring at least depth information.

15. The system as set forth in claim 10 wherein for the generate and transmit the one or more drive system controls based on the generated navigation path to a robotic drive system the one or more processors are further configured to be capable of executing the stored programmed instructions to:
generate and transmit one or more navigation arm system controls to the disinfecting arm control system to adjust positioning of one or more arms of the disinfection arm control system.

16. The system as set forth in claim 10 wherein for the adjust the one or more drive system controls to the robotic drive system further comprises:
receive navigation imaging data from one or more imaging devices during navigation of the generated navigation path;
identify any obstacles in the navigation imaging data during the navigation;
determine when any of the identified obstacles during the navigation require an alteration of the generated navigation path;
create updated layout data of the environment with a stored relative position of the identified objects; and
adjust the one or more drive system controls to the robotic drive system based on the updated layout data of the environment with the stored relative position of the identified objects to create one or more alterations to the navigation path when the determination indicates an alteration of the generated navigation path is required.

17. The system as set forth in claim 16 wherein the one or more imaging devices comprise an imaging and depth sensing system with at least one LIDAR and one or more cameras that capture imaging and depth sensing data; and
wherein at least one of the adjusting the one or more drive system controls to the robotic drive system or generating and transmitting one or more navigation arm system controls to the disinfecting arm control system to adjust positioning of one or more arms of the disinfection arm control system is based on the imaging and depth sensing data.

18. The system as set forth in claim 10 wherein the one or more processors are further configured to be capable of executing the stored programmed instructions to:
monitor the targeted disinfection to the one of the determined areas; and
determine when the targeted disinfection to the one determined areas is completed;
wherein the generate and transmit the one or more drive system controls further comprises instructions to generate and transmit one or more additional drive controls to navigate the generated navigation path to a next one of the determined areas in the environment when the determination indicates the targeted disinfection to the one areas is completed.

19. A non-transitory computer readable medium having stored thereon instructions comprising executable code which when executed by one or more processors, causes the one or more processors to:
determine one or more areas in an environment to target disinfection;
generate a navigation path based on at least layout data of the environment and the one or more determined areas in the environment to target disinfection;
generate and transmit one or more drive system controls based on the generated navigation path to a robotic drive system;
adjust the one or more drive system controls to the robotic drive system based on any obstacles during navigation that require an alteration of the generated navigation path; and
initiate one or more disinfection arm system controls to guide positioning of a disinfection arm system to one of the areas to target disinfection when the one or more drive system controls have positioned the disinfecting arm system adjacent to the one of the areas.

20. The non-transitory computer readable medium as set forth in claim 19 wherein the executable code when executed by the one or more processors further causes the one or more processors to:
obtain at least one of monitored traffic data or monitored physical contact data in the environment; and
process the at least one of the monitored traffic data or the monitored physical contact data in the environment to identify the one or more determined areas in the identified environment to disinfect.

21. The non-transitory computer readable medium as set forth in claim 20 wherein the executable code when executed by the one or more processors further causes the one or more processors to:
obtain imaging data from at least one of the imaging devices during the navigation; and
process the imaging data from to at least one of identify or update the one or more determined areas.

22. The non-transitory computer readable medium as set forth in claim 21 wherein for the process the imaging data to at least one of identify or update the one or more determined areas, the executable code when executed by the one or more processors further causes the one or more processors to:
execute an artificial intelligence target area identification algorithm based on the obtained imaging data to at least one of identify or update the one or more determined areas;
wherein the artificial intelligence target area identification algorithm is trained based on prior stored imaging data and associated determined areas in at least one of the environment or one or more other environments determined to be comparable to the environment based on one or more factors.

23. The non-transitory computer readable medium as set forth in claim 21 wherein the at least one of the imaging devices comprises at least one camera mounted on the disinfecting arm system and capable of measuring at least depth information.

24. The non-transitory computer readable medium as set forth in claim 19 wherein for the generate and transmit the one or more drive system controls based on the generated navigation path to a robotic drive system, the executable code when executed by the one or more processors further causes the one or more processors to:
generate and transmit one or more navigation arm system controls to the disinfecting arm control system to adjust positioning of one or more arms of the disinfection arm control system.

25. The non-transitory computer readable medium as set forth in claim 19 wherein for the adjust the one or more drive system controls to the robotic drive system, the executable code when executed by the one or more processors further causes the one or more processors to:
receive navigation imaging data from one or more imaging devices during navigation of the generated navigation path;
identify any obstacles in the navigation imaging data during the navigation;
determine when any of the identified obstacles during the navigation require an alteration of the generated navigation path;
create updated layout data of the environment with a stored relative position of the identified objects; and
adjust the one or more drive system controls to the robotic drive system based on the updated layout data of the environment with the stored relative position of the identified objects to create one or more alterations to the navigation path when the determination indicates an alteration of the generated navigation path is required.

26. The non-transitory computer readable medium as set forth in claim 25 wherein the one or more imaging devices comprise an imaging and depth sensing system with at least one LIDAR and one or more cameras that captures imaging and depth sensing data; and
wherein at least one of the adjusting the one or more drive system controls to the robotic drive system or generating and transmitting one or more navigation arm system controls to the disinfecting arm control system to adjust positioning of one or more arms of the disinfection arm control system is based on imaging and depth sensing data captured by the one or more imaging devices comprising an imaging and depth sensing system with at least one LIDAR and one or more cameras.

27. The non-transitory computer readable medium as set forth in claim 19 wherein the executable code when executed by the one or more processors further causes the one or more processors to further comprises:
monitor the targeted disinfection to the one of the determined areas; and
determine when the targeted disinfection to the one determined areas is completed;
wherein the generate and transmit the one or more drive system controls further comprises instructions to generate and transmit one or more additional drive controls to navigate the generated navigation path to a next one of the determined areas in the environment when the determination indicates the targeted disinfection to the one areas is completed.
